(19) United States
(12) Patent Application Publication
(10) Pub. No.: US 2011/0082393 A1
(43) Pub. Date: Apr. 7, 2011

(54) ORTHOPEDIC ORTHOSIS

(76) Inventor: Wolfgang BORT, (US)

(21) Appl. No.: 12/897,250

(22) Filed: Oct. 4, 2010

(30) Foreign Application Priority Data
Oct. 6, 2009 (DE) 10 2009 049 542.8

(51) Int. Cl. A61B 5/103; A61F 5/00

(52) U.S. Cl. 600/594; 602/19

(57) ABSTRACT

The invention relates to an orthopedic orthosis for the purpose of bracing a body region of a patient, which may include at least one sensor contrivance provided for detection of movements of said body region. According to the present invention, provision is made, in particular, for the orthosis to have at least one resiliently deformable sensor portion, which is disposed on the orthosis such that a movement of the body region causes resilient deformation of said sensor portion, that the sensor contrivance has at least one actor for coupling waves into the sensor portion and at least one sensor for sensing the waves coupled into the sensor portion, and that the actor and the sensor of the sensor contrivance are disposed on the sensor portion such that the sensor can sense the wave coupled into the sensor portion by the actor and changed according to the deformation of the sensor portion. The use thereof is for the detection of orthopedically detrimental movements of a patient.

### CROSS REFERENCE TO RELATED APPLICATIONS

This application claims priority under 35 U.S.C. §119 of German Patent Application No. 10 2009 049 542.8, filed Oct. 6, 2009, the entire disclosure of which is hereby incorporated by reference into this application.

### FIELD OF THE INVENTION

The invention relates to an orthopedic orthosis for bracing a body region of a patient. A generic orthosis comprises at least one sensor contrivance for the purpose of detecting the movement of said body region.

### BACKGROUND OF THE INVENTION

Orthopedic orthoses particularly serve to restore or maintain the functionality of damaged or highly stressed parts and regions of the body. They can be in the form of bandages that are predominantly made of a material of a flexible nature such as a textile material. Another variant of such orthopedic orthoses involves orthoses that are made, at least partly, of rigid components that are deformable only to a limited extent.

It is known, for example from DE 10 2007 003 515 A1, to provide sensors on an orthopedic orthosis for the purpose of detecting the movement of the body part to be braced and of indicating an orthopedically detrimental movement to the patient, optionally by means of a signal.

### SUMMARY OF THE INVENTION

It is an object of the invention to develop an orthosis of the generic kind, particularly a spinal orthosis, to the effect that the movement of the body region braced by the orthosis can be detected in a particularly reliable manner.

This object may be achieved according to the invention in that the orthosis may include at least one resiliently deformable sensor portion that is disposed on the orthosis in such a way that a movement of the body region causes a resilient deformation of this sensor portion. The sensor contrivance further comprises at least one actor for coupling waves into the sensor portion and at least one sensor for detecting the waves that have been coupled into the sensor portion. The actor and the sensor in the sensor contrivance are disposed on the sensor portion in such a way that the sensor can sense the wave that is coupled by the actor into the sensor portion and altered to an extent depending on the degree of deformation of the sensor portion.

Thus, in the orthosis of the invention, provision is made for a preferably flat sensor portion made of a resiliently deformable material to be positioned such that it undergoes deformation as a result of the movement of the body region. This deformation of the sensor portion causes a state of stress in the sensor portion depending on the type and extent of the movement of the body region. The actor and the sensor in the sensor contrivance are provided for detecting this state of stress. The actor couples a wave into the sensor portion, which wave propagates in the sensor portion until it reaches the sensor in the sensor contrivance. For this purpose, the actor is formed such that it can be caused to oscillate when excited by an electronic system. The wave coupled into the sensor portion undergoes a change, particularly with regard to its amplitude, that is governed by the extent of deformation of the sensor portion, that is to say, by the nature of the state of stress prevailing in the sensor portion. The change in the wave can be detected by an electronic system in the bandage in that the wave produced by the actor is sensed by means of the sensor adapted to detect oscillations.

It is possible, in principle, to utilize all forms of wave propagations in the sensor portion, for example, also bulk waves that penetrate the sensor portion. However, it is also regarded as being advantageous when the actor is adapted to couple surface waves into the sensor portion, and when the sensor is adapted to record these surface waves. The advantage of the use of these surface waves is that they require less excitation energy for generation thereof together with a low operating voltage for the actors and thus alleviate the hazard posed to the patient by the electric current used. Furthermore, the lower energy required for coupling surface waves into the sensor portion is advantageous with regard to the battery operating time. The use of surface waves additionally allows the actor and the sensor to be disposed on the same side of the sensor portion, which can give rise to advantages in terms of design.

The actor and the sensor are functional terms for the purposes of the present invention. Apart from an embodiment in which the actor and the sensor are in the form of separate structural components, an embodiment comprising only one structural component suitable both for the emission and reception of waves is also feasible. Such structural components are referred to as transceivers.

Those components can be used as actor and sensor that are suitable for converting an electrical voltage, more particularly an alternating voltage, to mechanical oscillations and, respectively, mechanical oscillations to a signal that can be evaluated by an electronic system. In particular, the actor and/or the sensor can be in the form of a piezoelectric actor and a piezoelectric sensor respectively.

Due to the arrangement of the sensor portion and the integration of the sensor portion in the orthopedic orthosis, it is possible to predefine the type of movement of the body region that leads to a change in the wave and thus to a recording of a movement. Different body regions can be monitored by the use of a plurality of sensor contrivances comprising different sensor portions so that the evaluation allows for precise assessment of the movement of the patient.

Preferably, the sensor contrivances are evaluated continuously. In such a case, the transmitter of the sensor contrivance continuously couples waves into the sensor portion. However, it is also feasible to monitor the movement of the body region at intervals of a few hundredths or tenths of a second by means of wave pulses generated at specific intervals in time.

According to a development of the invention, the orthosis comprises at least one main portion that surrounds the body region such that a movement of the body region causes deformation of the main portion. The sensor portion is in the form, at least in portions, of an integral part of the main portion, and the sensor portion is in the form, at least in portions, of a supplementary portion attached to the main portion.

The main portion is preferably in the form of a single piece that surrounds the relevant part of the body such that movement of that region of the body results in deformation of the main portion. In the case of an orthosis, such a main portion can be in the form of a rigid component, but one that is resiliently deformable to a limited extent. In the case of a bandage, the main portion can also be non-resiliently flexible. A mainly rigid main portion is preferably supplemented by a covering, for example, a preferably padded textile covering in order to enhance wearing comfort.

In the case of a rigid but resiliently deformable main portion, a part of this main portion can directly form the sensor portion of the at least one sensor contrivance. This results in a very simple design.

However, the use of an additional portion that is attached to the main portion non-positively, positively or adhesively and is thus deformed along with a deformation of the main portion is regarded as being advantageous. The use of bonded and/or positive fasteners, particularly the use of rivets, for attaching the additional portion to the main portion is regarded as being particularly advantageous. The use of an additional portion that is separate from the main portion makes it possible to employ optimal materials for the main portion and the additional portion. Thus, for example, the main portion can be in the form of a component made of plastics material, more particularly one that is made of polyethylene, thereby keeping down the production costs. However, the additional portion comprising the sensor portion can be in a form such that it allows for perfectly resilient behavior, more particularly for behavior that is free from relaxation as far as possible, and can be made of a fiber composite component for this purpose. More particularly, carbon fiber reinforced plastics materials or carbon fiber reinforced epoxy resins are suitable for this purpose. When use is made of fiber-reinforced composites, the fibers must preferably be oriented such that they extend in the direction of the intended wave propagation from the actor to the sensor.

In the case of an embodiment comprising a separate additional portion disposed on the main portion, it is regarded as being advantageous when the main portion is provided with a recess in which the sensor and/or the actor is disposed, at least in part. This embodiment makes for a particularly flat design of the orthopedic orthosis since the sensor and/or the actor can be disposed on that side of the supplementary portion that faces the main portion. The arrangement of the sensor and/or the actor in the recess can additionally be utilized for attaching the additional portion to the main portion by means of a preferably bonded connection of the actor or sensor to the edge of the recess.

According to one variant of the relative arrangement of the actor and the sensor, provision is made for the actor and the sensor to be disposed at opposite ends of the sensor portion and to be connected to the same. It is regarded as being advantageous when a wave reflector is provided on the sensor portion, which wave reflector is adapted to reflect a wave coming from the actor such that it is directed toward the sensor. This makes it possible, in particular, to position the actor and the sensor in the sensor contrivance such that they are directly juxtaposed or even to use of an easy to handle single structural unit comprising both the actor and the sensor.

The actor and the sensor in the sensor contrivance are preferably disposed on the sensor portion in such a way that the wave coupled into the sensor portion by the actor travels through a distance of at least 5 mm before it reaches the sensor. In the case of a sensor and an actor that form a combined structural unit, the wave reflector is preferably disposed at a distance of at least 2.5 mm from this structural unit. Greater wave propagation distances of at least 10 mm between the actor and the sensor are preferred.

A boundary surface of the sensor portion that causes reflection of the wave is regarded as the wave reflector. Preferably, the wave reflector is in the form of a fastener such as a rivet that serves to attach the supplementary portion to the main portion and thus performs a dual function.

An orthopedic orthosis of the invention can serve to brace various regions and parts of the body and can thus be adapted for use on the knee joint or ankle joint or on the elbow joint or wrist joint. A neck orthosis can also be provided in the manner proposed by the invention. It is regarded as being particularly advantageous when the orthopedic orthosis is in the form of a spinal orthosis, the sensor contrivance being preferably disposed in a back region of this orthosis.

The detection of a state of stress in the sensor portion is preferably carried out by evaluating the wave attenuation caused by this state of stress. Accordingly, the excitation frequency of the actor can preferably be selected so as to prevent unduly strong attenuation and also an unintended increase in oscillation. It is regarded as being particularly advantageous when an electronic control system that is connected to the sensor contrivance is adapted to excite the actor at a frequency ranging from 10 kHz to 10 MHz. A particularly good signal-to-noise ratio is achieved at frequencies ranging from 25 kHz to 100 kHz and 220 kHz to 1 MHz.

It is advantageous when an orthopedic orthosis of the invention comprises a plurality of sensor contrivances. It is particularly advantageous when the respective sensor portions of at least two of these sensor contrivances are in the form of a single-piece supplementary portion attached to the main portion. The use of a combined supplementary portion, on which a plurality of sensor contrivances and thus a plurality of sensor portions are provided, results in a simpler design and, in particular, in reduced effort for attaching the supplementary portion to the main portion.

As a development of the aforementioned orthopedic orthosis, the invention also relates to a generic orthopedic orthosis that is in the form of a spinal orthosis comprising at least one back portion disposed in the region of the back of the patient and at least one pair of wing-like portions joined to the back portion, the pair of wing-like portions comprising two wing-like portions that are attached to the sides of the back portion and are disposed opposite each other and are bent around opposite sides of the body to extend to the chest and/or abdominal region of the patient. A plurality of sensor contrivances is provided that are adapted to detect elongations or states of stress in the orthopedic orthosis, at least one sensor contrivance of a first type being disposed on the back portion at the level of the pair of wing-like portions or on one of the wing-like portions, and at least one sensor contrivance of a second type being disposed on the back portion above or below the wing-like portions.

The spinal orthosis of the invention comprises an immovable back portion that is resiliently deformable to a limited extent and starting from which the wing-like portions extend to the chest and abdominal side of the patient. The wing-like portions are preferably formed integrally as a single piece with the back portion, but are at least attached to the same. The wing-like portions firstly serve to fasten the spinal orthosis to the body of the patient, for which purpose, they can preferably be fastened to each other in front of the abdominal or chest region of the patient by means of detachable fasteners such as belts and/or hook-and-loop fasteners. Secondly, the wing-like portions serve to identify any movement of the torso of the patient. For this purpose, the sensor contrivances of the first and the second type are provided.

When the patient turns the upper part of his body to the left or to the right, this movement results in a resilient displacement of the wing-like portions relatively to the back portion. This deformation can be ascertained by the detection of a corresponding elongation or a state of stress by the at least one sensor contrivance of the first type. For this purpose, the latter is disposed either on the wing-like portion itself or in a transition region between a wing-like portion and the back portion, which transition region is partially deformed in the case of a resilient displacement of the wing-like portion in relation to the back portion. For detecting a forward bending movement or a backward bending movement of the patient, the at least one sensor contrivance of the second type is provided such that it is disposed above or below the wing-like portion. When the patient bends forward or backward, a deformation of the back portion of the orthosis is caused, at least in part, by his back or his chest or abdomen via the wing-like portions, which deformation can be detected by said sensor contrivance of the second type.

The aforementioned feature involving the attachment of the sensor contrivances makes it possible to effectively distinguish between a forward/backward bending movement and a torsional movement. A forward/backward bending movement hardly causes stresses or elongations in the region of the sensor contrivance of the first type due to the increased mechanical section modulus in the region of the sensor contrivance arising from the presence of the wing-like portions. Instead, such a movement is detected primarily by the sensor contrivance of the second type that is disposed above or below the wing-like portions. Conversely, a torsional movement of the upper body of the patient substantially causes only a state of stress or an elongation at the level of the wing-like portions and thus in the region of the sensor contrivance of the first type, while the sensor contrivance of the second type is hardly influenced thereby.

Thus the sensor contrivance of the first type preferentially detects a bending deformation of the orthosis in the region of the wing-like portions, while the sensor contrivance of the second type preferentially detects a bending deformation in the region of the back portion.

Instead of only one back portion, a plurality of back portions can be provided that are joined together by belts, for example. The back portion is preferably in the form of a single piece or it comprises a plurality of components that are rigidly attached to each other.

The sensor contrivances are preferably sensor contrivances of the embodiment described above comprising an actor, a sensor, and a sensor portion into which the actor can couple a wave that can be detected by the sensor in an altered form depending on the degree of deformation. It is a particular advantageous that sensor contrivances of identical design can be used as sensor contrivances of the first and second types, since both the torsional movement of the patient and the forward/backward bending movements are each manifested as detectable states of stress. It is possible to operate the different actors of the sensor contrivances using either different frequencies or the same frequency. The latter makes it possible to connect the actors in parallel, which is particularly advantageous with regard to the conductor layout.

Apart from sensor contrivances based on wave evaluation, it is also possible to use other sensors, particularly those adapted for distance measurement such as strain gauges or optical distance sensors.

Preferably, two sensor contrivances of the first type are provided which are each assigned to a wing-like portion of a combined pair of wing-like portions. The sensor contrivances can be provided as described above either in the transition region between the back portion and the wing-like portion or on the wing-like portion itself. By means of the embodiment comprising two such sensor contrivances of the first type, it is possible to detect the direction in which a patient turns the upper part of his body.

Furthermore, preferably two pairs of wing-like portions that are vertically spaced from each other are provided, and preferably at least two sensor contrivances of the first type and at least two pairs of sensor contrivances of the first type are provided, each of which is assigned to one of the pairs of wing-like portions. The use of two pairs of wing-like portions is advantageous, particularly since the at least one sensor contrivance of the second type can very reliably identify a forward bending movement of the patient, because the pairs of wing-like portions disposed at a distance from each other result in a bending deformation of the back portion when the patient bends forward. An embodiment comprising sensor contrivances of the first type that are each provided on the pairs of wing-like portions permits particularly reliable detection of a torsional movement of the upper part of a patient's body.

Furthermore, it is regarded as being particularly advantageous when at least two sensor contrivances of the second type are provided on the back portion, of which a first sensor contrivance is disposed in the vertical direction between two pairs of wing-like portions and of which a second sensor contrivance is disposed above the top pair of wing-like portions or below the bottom pair of wing-like portions. This embodiment comprising at least two sensor contrivances of the second type that are offset from each other in the vertical direction is advantageous with regard to the ability to distinguish between a forward bending movement and a backward bending movement. While with a backward bending movement, all sensor contrivances of the second type detect an elongation or a state of stress due to the deformation of the back of the patient, this applies to the forward bending movements, when the sensor contrivances are disposed between the pairs of wing-like portions. The sensor contrivance of the second type that is disposed above the top pair of wing-like portions or below the bottom pair of wing-like portions experiences no influence or experiences a significantly weaker influence during the forward bending movement so that this second sensor contrivance thus disposed makes it possible to reliably distinguish between a forward bending movement and a backward bending movement.

An orthosis of the invention preferably comprises an electronic evaluation unit that is connected to the at least one sensor contrivance or the at least two sensor contrivances and that indicates an orthopedically detrimental movement of the body region by way of an indicating means dependent on the signals arising from this sensor contrivance. For this purpose, the electronic evaluation unit evaluates the signals coming from the sensor contrivances and draws conclusions on the movement carried out by the patient on the basis of the signal or, in the case of a plurality of sensor contrivances, on the basis of the plurality of signals. In the simplest case, this movement is classified as being orthopedically permissible or orthopedically detrimental. An orthopedically detrimental movement gives rise to the emission of a patient information signal by the indicating means, for example in the form of a warning signal or a vibration. The electronic evaluation unit may be adapted to store some or all of the characteristics of the movements performed by the patient in a memory so that an orthopedist can subsequently evaluate these data.

In the context of the sensor contrivances based on wave analysis, the evaluation preferably involves the analysis of wave attenuation and/or wave travel time. In order to facilitate the analysis of wave attenuation, a circuit can be disposed upstream of the electronic evaluation unit, which circuit converts the signal originating from the sensor contrivance to a transformed signal, the strength of which is governed by the amplitude of the signal originating from the sensor contrivance. Such a circuit can, for example, be of such a kind that it converts the signal originating from the sensor contrivance and then passes it through a low-pass filter. Such signal conversion reduces the complexity of the electronic evaluation unit, particularly the power required by a microprocessor in the electronic evaluation unit, since it is not the actual wave detected by the sensor but the amplitude thereof that is to be analyzed by the electronic evaluation unit. An analysis of the signal amplitude is sufficient for determining the respective attenuation of the wave as results from the state of stress in the sensor portion.

The respective thresholds, based on which the electronic evaluation unit identifies an orthopedically detrimental state and optionally indicates the same by way of the indicating means, can be fixed or can be selectively adjustable. However, it is particularly advantageous when the electronic evaluation unit can be transferred to an initialization mode in which the thresholds are formed as a function of the signals coming from the sensor contrivance, the electronic evaluation unit being adapted to utilize these thresholds in a subsequent operating mode for identifying orthopedically detrimental movements of the relevant body region. The initialization mode, which can be started, for example, by depressing a special press-button disposed on the orthosis, makes it possible to customize the orthosis to the needs of each patient in a convenient manner. In the initialization mode, the patient moves, optionally as instructed by the operating manual delivered with the orthopedic orthosis, or under the supervision of an orthopedist or as directed by the control unit itself, in such a way that the electronic evaluation unit can detect the signals coming from the sensors as produced by predefined movements of the specific region or part of the body. The thresholds determined on this basis are thus individually customized to the patient.

### BRIEF DESCRIPTION OF THE DRAWINGS

Additional aspects and advantages of the invention are revealed in the claims and in the following-like description of a preferred exemplary embodiment of the invention explained with reference to the figures, in which:

FIGS. 1 to 3 show a spinal orthosis in a back and front view and a sectional view taken from above, respectively,

FIGS. 4a to 4c illustrate the mode of operation of the sensor contrivances of the spinal orthosis shown in FIGS. 1 to 3, and

FIGS. 5a to 5d illustrate the use of the spinal orthosis shown in FIGS. 1 to 3 and those influences on the sensor contrivances of the orthosis that accompany different movements of a patient.

### DETAILED DESCRIPTION OF THE INVENTION

FIGS. 1 to 3 show a spinal orthosis 10 of the invention.

The spinal orthosis 10 comprises a main portion 20 that is in the form of a single-piece flat plastics component made of polyethylene or some other plastics material. This main portion 20 comprises a back portion 22 on which a top pair of wing-like portions 24 comprising wing-like portions 24a and 24b and a bottom pair of wing-like portions 26 comprising wing-like portions 26a and 26b are provided at the sides. These wing-like portions 26a, 26b, 24a, and 24b are molded onto the two opposite sides of the back portion 22 and they extend arcuately toward the front.

The main portion 20 of the spinal orthosis 10 is completely surrounded by a textile covering 12 indicated by dashed lines in FIGS. 1 to 3. It serves, in particular, to enhance the wearing comfort.

On the front side of the spinal orthosis 10, the wing-like portions 24b and 26b are each attached to fastening belts 14, on the free ends of which hook-and-loop surfaces 16a are provided that are each intended for cooperation with corresponding hook-and-loop surfaces 16b disposed on wing-like portions 24a and 26a, thereby allowing-like the wing-like portions 24a, 24b, 26a, and 26b to be joined together in front of the abdomen or chest of the patient.

Two flat bars 40 that are made of carbon fiber reinforced plastics material and that extend substantially in the vertical direction are provided on each side of the back portion 22, and these flat bars are joined to the main portion 20 by rivets 44. These flat bars 40 are provided on the inside surface of the back portion 22, but are covered by the textile covering 12 such that they are not visible to the patient.

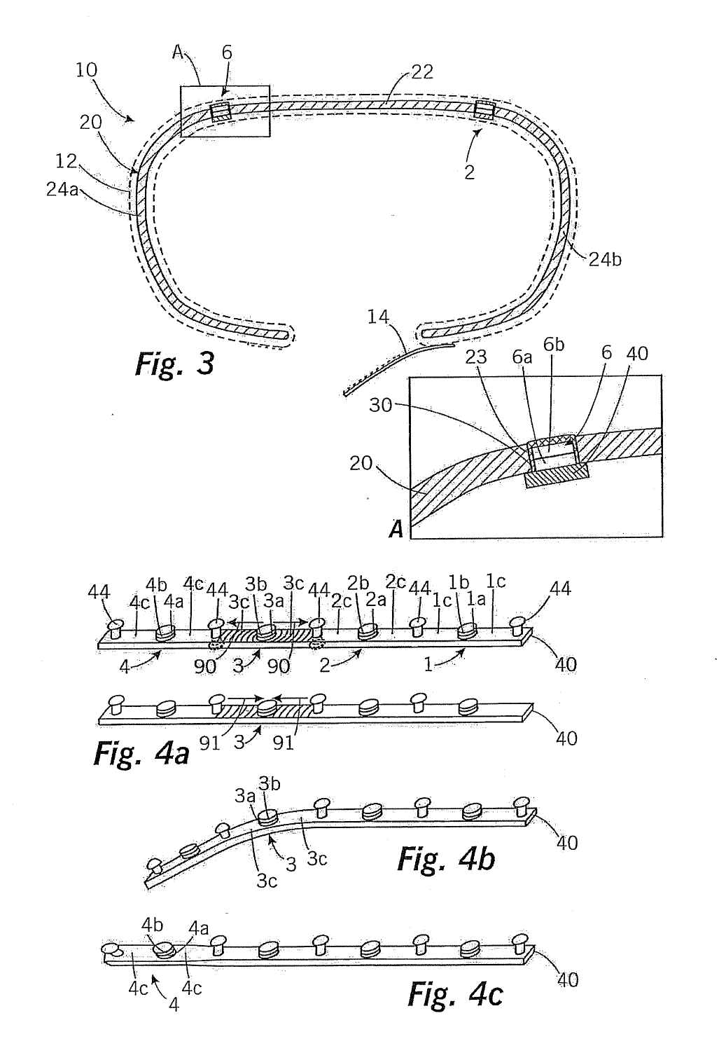

The flat bars 40 serve as supports for sensor contrivances 1, 2, 3, 4, 5, 6, 7, and 8. Referring to FIG. 3, in particular, and the portion A shown on an enlarged scale in FIG. 3, the sensor contrivances each comprise an actor 1a to 8a and a sensor 1b to 8b. These actors 1a to 8a and sensors 1b to 8b are disposed such that they are directly juxtaposed. Furthermore, the actors 1a to 8a are directly attached to the flat bars 40, for example by means of an adhesive bond. The actors 1a to 8a are in the form of piezoelectric actors that can oscillate at high frequencies when appropriately excited. The sensors 1b to 8b are in the form of piezoelectric sensors and are adapted to be capable of detecting oscillations and converting the same to an electrical signal that can be analyzed.

As is evident from the detail A shown in FIG. 3, the actors 1a to 8a and the sensors 1b to 8b are disposed in recesses 23 of the back portion 22 of the main portions 20. They are attached by means of an adhesive 30 so that the flat bars 40 are physically connected to the main portion 20 not only by means of the rivets 44 but also by means of the sensors 1b to 8b and the actors 1a to 8a and the adhesive 30.

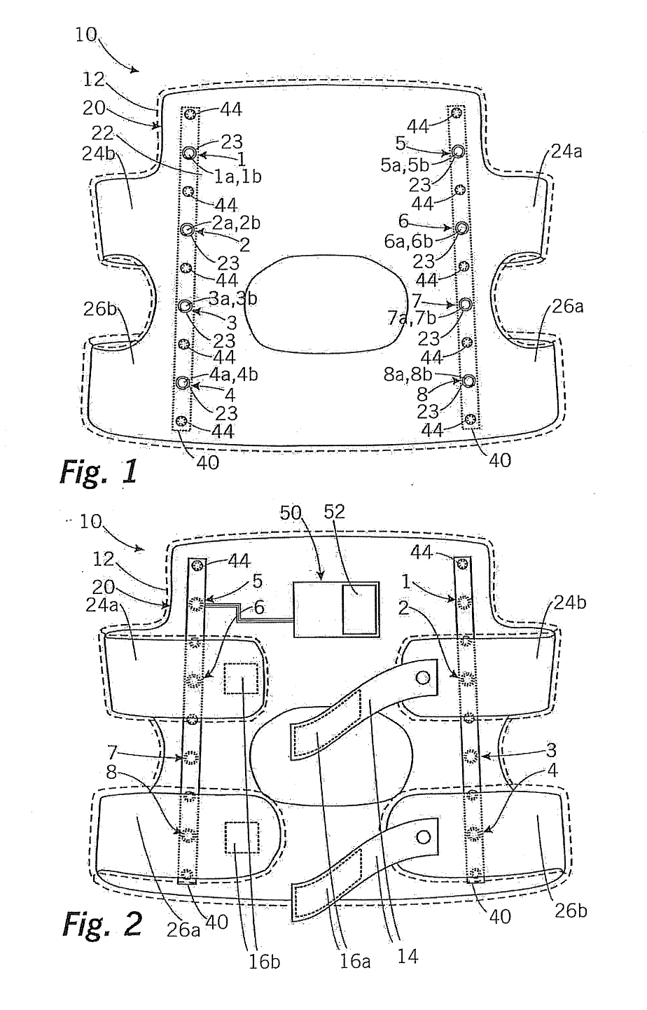

An electronic control/evaluation unit 50 is further disposed on the back portion 22 on the inside surface thereof such that the electronic control/evaluation unit is hidden under the textile covering 12. The electronic control/evaluation unit 50 is connected to the actors 1a to 8a and the sensors 1b to 8b by means of electrical conductors. This is shown merely by way of example for the sensor contrivance 5 in FIG. 2. The connection of the other sensor contrivances to the control unit 50 is similarly configured.

The control unit 50 is adapted to supply the actors 1a to 8a with a high-frequency alternating voltage in order to cause the actors to oscillate. A frequency of 500 kHz is used for this purpose. Furthermore, the control unit 50 is adapted to evaluate the oscillations detected by the sensors 1b to 8b.

The mode of operation of the sensor contrivances 1 to 8 is explained below with reference to FIGS. 4a to 4c. The mode of operation is explained with reference to the sensor contrivances 1 to 4 shown on the left hand side in FIG. 1.

As explained above, the sensor contrivances 1 to 4 are provided on one of the two flat bars 40 made of carbon fiber reinforced plastics material. A sensor portion 1c, 2c, 3c, and 4c is assigned to each set comprising one actor of the actors la to 8a and one sensor of the sensors 1b to 8b. These sensor portions 1c, 2c, 3c, and 4c are each sub-portions of the flat bar 40, each sensor portion being delimited on both sides by rivets 44. The main portion 20 disposed above the flat bar 40 is not shown in FIGS. 4a to 4c.

The mode of operation of the sensor contrivances 1 to 8 is explained with reference to FIG. 4a and exemplified by the sensor contrivance 3. The actor 3a is excited by the control unit 50 with an alternating voltage having the aforementioned frequency of 500 kHz. The resulting oscillation of the actor 3a leads to coupling of waves into the sensor portion 3c. The waves propagate in both directions 90 until they are reflected by the rivets 44 in the direction 91. The reflected waves are detected by the sensor 3b and transmitted to the control unit 50 in the form of a corresponding signal. The control unit 50 can compare the recorded wave with the wave coupled into the sensor portion and accordingly draw conclusions on the state of stress in the sensor portion 2c, for example, on the basis of the attenuation of the waves.

In the state shown in FIG. 4a, the flat bar 40 is in the relaxed state so that the attenuation is low.

In the state shown in FIG. 4b, the sensor portion 3c of the sensor contrivance 3 is bent as a result of a force applied to the flat bar 40, and the surface of sensor portion 3c is thus under tensile stress. The waves coupled into the sensor portion 3c by the actor 3a are attenuated to a greater degree before they are detected by the sensor 3b, due to this state of stress. The control unit 50 is thus able to identify the state of stress and thus the deformation of the sensor portion 3c on the basis of the increased attenuation.

The flat bar 40 is twisted in the region of sensor portion 4c in the state shown in FIG. 4c. This also leads to a state of stress that causes the wave coupled by the actor 4a into the sensor portion 4c to reach the sensor 4b at a degree of attenuation which is greater than that occurring in the stress-free state shown in FIG. 4a.

Although the states of stress of the sensor portion 3c shown in FIG. 4b and the sensor portion 4c shown in FIG. 4c are caused by a bent flat bar and a twisted flat bar respectively, they can be identified by sensors/actors of identical design since both types of stress states bring about increased attenuation.

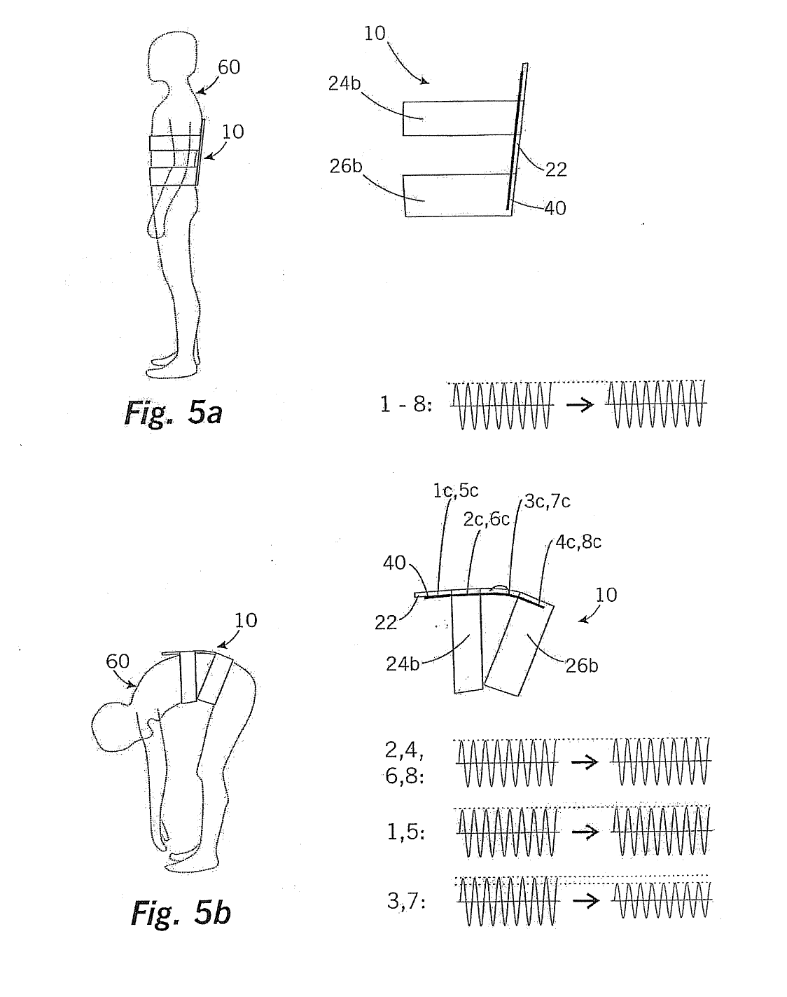

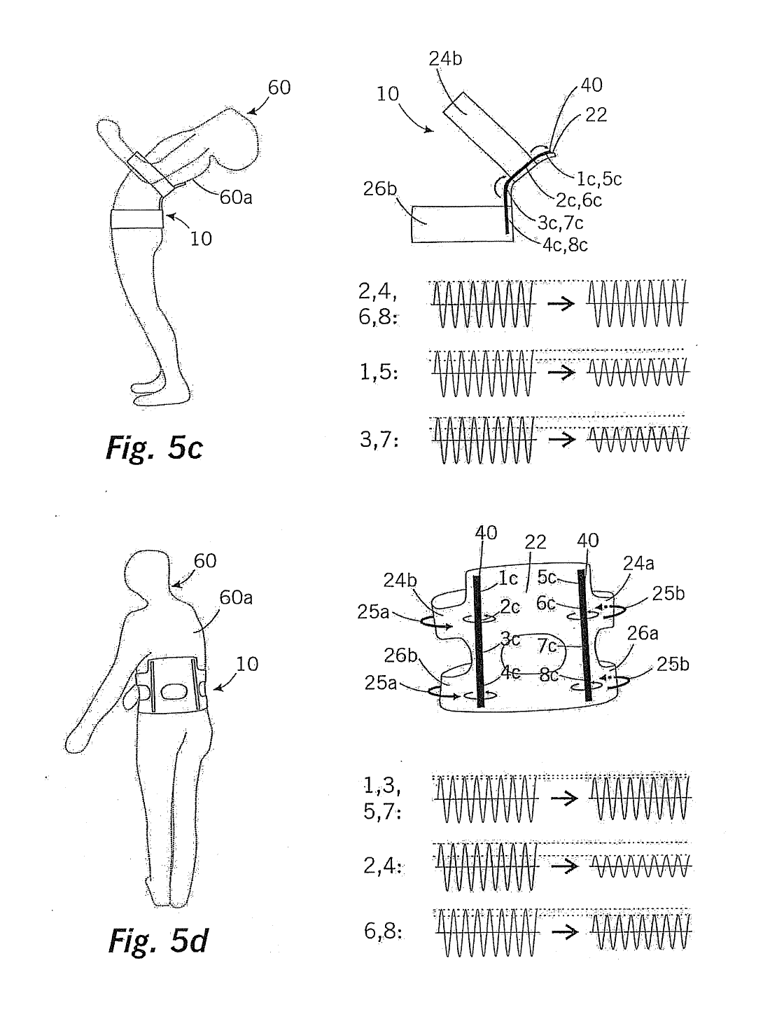

The movement-specific deformation of the flat bars 40 in the case of a deformation of the main portion 22 is utilized to enable the control unit 50 to recognize and distinguish between various movements of the patient. The manner in which this takes place is explained below in a simplified manner with reference to FIGS. 5a to 5d.

FIG. 5a shows an initial state of the spinal orthosis 10. The patient 60 maintains an upright posture. In this state, the flat bars 40 are relaxed as shown in the illustration of FIG. 4a. As a result, the coupled wave shown on the right-hand side of FIG. 5a reach the respective sensors 1b to 8b almost without any attenuation as is evident from the representations of oscillations shown in FIG. 5a. On the basis of this condition, the control unit can identify the upright posture of the patient 60.

FIG. 5b shows the patient 60 bent forward. Due to the wing-like portions 24a, 24b, 26a, and 26b, this posture causes the flat bars 40 to bend in the region of the sensor portions 3c and 7c of the sensor contrivances 3 and 7, respectively. The remaining sensor portions 1c, 2c, 4c, 5c, 6c, and 8c are not bent or are hardly bent. In the case of the sensor portions 1c and 5c, this is due to the fact that they are provided above the top wing-like portions 24a and 24b so that the wing-like portions 24a, 24b, 26a, 26b cannot produce any bending moment at this location. The fact that the sensor portions 2c, 4c, 6c, and 8c are not bent or are hardly bent is due to the increased mechanical section modulus of the back portion 22 relative to bending in the region of these sensor portions caused by the presence of the wing-like portions 24a, 24b, 26a, and 26b. As shown by the wave diagrams in FIG. 5b, an increased attenuation can therefore be observed only in the region of the sensor contrivances 3 and 7. Such an attenuation confined to the sensor contrivances 3 and 7 can thus be clearly identified by the control unit 50 as being an indication of the fact that the patient is bending forward.

FIG. 5c shows the patient 60 bending back. This posture also causes bending of the back portion 22 and thus of the flat bars 40. However, this is caused, unlike the forward bending posture shown in FIG. 5b, by the posture of the back 60a of the patient so that in contrast to the forward bend shown in FIG. 5b, the sensor contrivances 1 and 5 record an increased degree of attenuation. On the other hand, the sensor contrivances 2, 4, 6, and 8 remain largely unaffected due to the increased section modulus of the back portion 22 due to the presence of the wing-like portions 24a, 24b, 26a, and 26b. The attenuation detected can thus be clearly assigned by the control unit 50 to a backward bending movement.

FIG. 5d shows a position of the patient, in which the latter has turned the upper part of his body toward the left. Such a movement causes the left-hand wing-like portions 24b and 26b to bend back in the direction of the arrows 25a. At the same time, the right-hand wing-like portions 24a and 26a on the front side of the patient are pulled toward the left by means of the fastening belt 14 so that they are bent forward in the direction of the arrows 25b. This bending stress of the wing-like portions 24a, 24b, 26a, and 26b results in each of the flat bars 40 being partially twisted in the direction of the arrows drawn in the region of the sensor portions 2c, 4c, 6c, and 8c so that the sensor contrivances 2, 4, 6, and 8 record an increased degree of attenuation. Due to the fact that this torsion is stronger in the rotational direction, that is to say, toward the left rather than to the right, the control unit 50 can detect that the patient has moved his body toward the left.

It is apparent from the explanation of FIGS. 5a to 5d that the control unit 50 can reliably detect the movement of the patient. The attenuation factor enables a detection of not only the type but also the extent of the respective movements. The control unit 50 can compare the determined movement or posture of the patient with predefined thresholds in order to assess whether the movement or posture in question is one that is orthopedically detrimental. When this is the case, the control unit 50 can inform the patient by means of an integrated vibration signal transmitter 52 to the effect that his present movement or posture should be avoided.

What is claimed is:

1. An orthopedic orthosis for bracing a body region of a patient, comprising
at least one sensor contrivance provided for sensing the motion of said body region,
wherein
said orthosis comprises at least one resiliently deformable sensor portion, which is disposed on the orthosis such that a movement of the body region causes resilient deformation of said sensor portion,
said sensor contrivance has at least one actor for coupling waves into the sensor portion and at least one sensor for detecting the waves coupled into the sensor portion, and
said actor and said sensor in said sensor contrivance are disposed on said sensor portion such that said sensor can sense the wave which has been coupled into the sensor portion by said actor and has been changed according to the deformation of said sensor portion.

2. The orthopedic orthosis as defined in claim 1, wherein
said orthosis has at least one main portion which surrounds the body region such that a movement of the body region causes deformation of said main portion, and
said sensor portion is formed at least in part as an integral part of said main portion and/or said sensor portion is formed at least in part by a supplementary portion mounted on said main portion.

3. The orthopedic orthosis as defined in claim 2, wherein
said main portion is a part made of plastics material, particularly a part made of polyethylene, and/or the supplementary portion is made of fiber-reinforced compound material.

4. The orthopedic orthosis as defined in claim 2, wherein
in said main portion there is provided a recess, in which recess said sensor and/or said actor are disposed at least partially, said actor or said sensor being cast or bonded in said recess.

5. The orthopedic orthosis as defined in claim 1, wherein
a wave reflector is provided on the sensor portion and is adapted to reflect a wave coming from said actor toward said sensor, said actor and said sensor being directly juxtaposed in said sensor contrivance.

6. The orthopedic orthosis as defined in claim 1, wherein
said orthosis is in the form of a spinal orthosis, the sensor contrivance being disposed on a back region of said orthosis.

7. The orthopedic orthosis as defined in claim 1, wherein
control electronics connected to said sensor contrivance, said control electronics being adapted to excite said actor at a frequency of from 10 kHz to 10 MHz.

8. The orthopedic orthosis as defined in claim 1, wherein
a plurality of sensor contrivances is provided, and the respective sensor portions contained in at least two such sensor contrivances are formed by an integrated supplementary portion and are mounted on said main portion.

9. The orthopedic orthosis as defined in claim 1, wherein
said orthosis is in the form of a spinal orthosis having at least one back portion disposed in the back region of the patient and at least one pair of wing-like portions connected to said back portion and said pair of wing-like portions consists of two wing-like portions which are provided opposite to each other on each side of said back portion and are bent around the opposite side of the body to extend to the region of the chest and/or abdomen, and
a plurality of sensor contrivances is provided which are adapted to detect changes in length or states of stress in the orthopedic orthosis, and
at least one sensor contrivance of a first type is disposed on the back portion at the level of said pair of wing-like portions or on one of said wing-like portions, and
at least one sensor contrivance of a second type is disposed on said back portion below or above said pair of wing-like portions.

10. The orthopedic orthosis as defined in claim 9, wherein
two sensor contrivances of the first type are provided, each of which is assigned to a wing-like portion of a common pair of wing-like portions.

11. The orthopedic orthosis as defined in claim 9, wherein
two vertically spaced pairs of wing-like portions are provided, and
at least two sensor contrivances of the first type or at least two pairs of sensor contrivances of the first type are provided, each of which is assigned to one of the pairs of wing-like portions.

12. The orthopedic orthosis as defined in claim 9, wherein
at least two sensor contrivances of the second type are provided on the back portion, of which a first sensor contrivance is vertically disposed between two pairs of wing-like portions and of which a second sensor contrivance is disposed above the top pair of wing-like portions or below the bottom pair of wing-like portions.

13. The orthopedic orthosis as defined in claim 1, wherein
an electronic evaluation circuit is provided which is connected to said at least one sensor contrivance and, depending on the signals emitted by said at least one sensor contrivance, indicates an orthopedically detrimental movement of the body region by means of a signaling device.

14. The orthopedic orthosis as defined in claim 13, wherein
upstream of said electronic evaluation circuit there is provided a circuit which converts the signal coming from said sensor contrivance to a transformed signal whose signal strength depends of the amplitude of the signal coming from said sensor contrivance.

15. The orthopedic orthosis as defined in claim 13, wherein
the electronic evaluation circuit can be switched to an initialization mode in which limiting values are formed which depend on signals coming from said sensor contrivances, said electronic evaluation circuit being adapted to utilize the limiting values in a subsequent operation mode for the recognition of orthopedically detrimental movements of said body region.
